(19) United States
(12) Patent Application Publication
(10) Pub. No.: US 2020/0030472 A1
(43) Pub. Date: Jan. 30, 2020

(54) Bidet Having Bidet Seat Sterilizing Function

(71) Applicant: COWAY CO., LTD., Chungcheongnam-do (KR)

(72) Inventors: Keun-Hwan Kim, Seoul (KR); Man-Uk Park, Seoul (KR); Sung-Worl Jin, Seoul (KR); Doo-Youl Jeon, Seoul (KR); Da-Woon Jung, Seoul (KR); Ji-Won Han, Seoul (KR)

(21) Appl. No.: 16/491,203

(22) PCT Filed: Apr. 5, 2018
(86) PCT No.: PCT/KR2018/004032

§ 371 Date: Sep. 5, 2019

(30) Foreign Application Priority Data
Apr. 5, 2017 (KR) 10-2017-0044153

(51) Int. Cl. A61L 2/10; E03D 9/08; A61L 2/26

(52) U.S. Cl. CPC A61L 2/10; E03D 9/08; A61L 2202/14; A61L 2202/11; A61L 2/26

(57) ABSTRACT

Disclosed is a bidet having a bidet seat sterilizing function. The bidet having a bidet seat sterilizing function may include: a bidet seat, disposed on the upper portion of a toilet seat, for seating a user thereon; a bidet cover for opening and closing the bidet seat; a sterilizing part which is installed on the bidet seat-facing surface of the bidet cover and sterilizes the bidet seat when the bidet cover covers the bidet seat; a driving part for driving the bidet seat and the sterilizing part; and a control part for controlling the sterilizing part and the driving part.

### TECHNICAL FIELD

The present disclosure relates to a bidet having a bidet seat sterilizing function using an ultraviolet light emitting diode (UVLED).

### BACKGROUND ART

In general, a bidet is a device for cleaning the anus of a user or the pudendum of female by spraying cleaning water from a nozzle without the need for the use of toilet paper, after the user has relieved him or herself, and then drying the water remaining around the anus or the pudendum with a warm air supplied from a dryer. Recently, a bidet has been widely used by both sexes as interest in personal hygiene has increased.

The bidet is inevitably in physical contact with the bidet seat when used, and, as the number of times of physical contact increases, the bidet seat is highly likely to become contaminated, such that periodic sterilization is required.

There has been proposed a method of sterilizing or disinfecting a bidet seat by additionally installing an ultraviolet lamp. However, there is a problem in which a lifetime of an ultraviolet lamp is shortened as well as a cost is increased due to power consumption, due to continuous exposure of the ultraviolet lamp for sterilization and disinfection.

Further, due to a constant irradiation time, there is a problem in which a contamination state may not be completely eradicated, depending on the distance.

### DISCLOSURE

### Technical Problem

An aspect of the present disclosure is to provide a bidet having a bidet seat sterilizing function for measuring a usage pattern of a user and determining a sterilizing time zone.

An aspect of the present disclosure is to provide a bidet having a bidet seat sterilizing function for measuring a distance from the bidet seat and determining an irradiation time according to the measured distance to irradiate ultraviolet rays.

### Technical Solution

According to an aspect of the present disclosure, a bidet having a bidet seat sterilizing function is provided.

The bidet having a bidet seat sterilizing function may include: a bidet seat, disposed on an upper portion of a toilet seat, allowing a user to be seated thereon; a bidet cover for opening and closing the bidet seat; a sterilizing part which is installed on the bidet seat-facing surface of the bidet cover and sterilizes the bidet seat when the bidet cover covers the bidet seat; a driving part for driving the bidet seat and the sterilizing part; and a control part for controlling the sterilizing part and the driving part.

Further, a solution to the above-mentioned problems does not list all possible features of the present disclosure. The various features and advantages and effects of the present disclosure can be understood in more detail with reference to the following specific embodiments.

### Advantageous Effects

An automatic sterilizing operation at an unused time of the bidet by measuring a usage pattern of a user may be periodically performed.

In addition, a sterilizing effect may be increased by measuring a distance from the bidet seat and determining a sterilization time according to the measured distance.

### DESCRIPTION OF DRAWINGS

FIG. 1 is a front view of a bidet having a bidet seat sterilizing function according to an embodiment of the present disclosure.

FIG. 2 is a side view of a bidet having a bidet seat sterilizing function according to an embodiment of the present disclosure.

FIG. 3 is a configuration diagram of a bidet having a bidet seat sterilizing function according to an embodiment of the present disclosure.

FIG. 4 is a flow chart of the control part of FIG. 3.

### BEST MODE

Hereinafter, exemplary embodiments of the present disclosure will be described in detail with reference to the accompanying drawings. The disclosure may, however, be exemplified in many different forms and should not be construed as being limited to the specific embodiments set forth herein, and those skilled in the art and understanding the present disclosure can easily accomplish retrogressive inventions or other embodiments included in the scope of the present disclosure by the addition, modification, and removal of components within the same scope, but those are construed as being included in the scope of the present disclosure. Like reference numerals will be used to designate like components having similar functions throughout the drawings within the scope of the present disclosure.

Throughout the specification, a configuration is referred to as being ‘connected’ to another configuration, including not only when the configurations are directly connected but also when they are indirectly connected with each other. In addition, when a component is referred to as “comprise” or “comprising,” it means that it may include other components as well, rather than excluding other components, unless specifically stated otherwise.

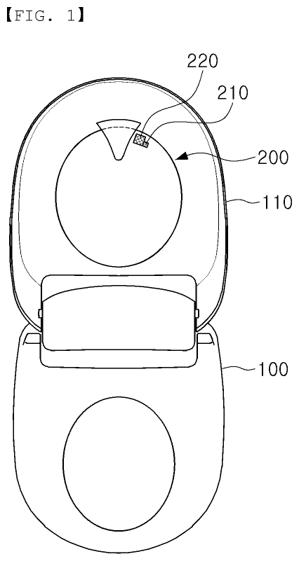

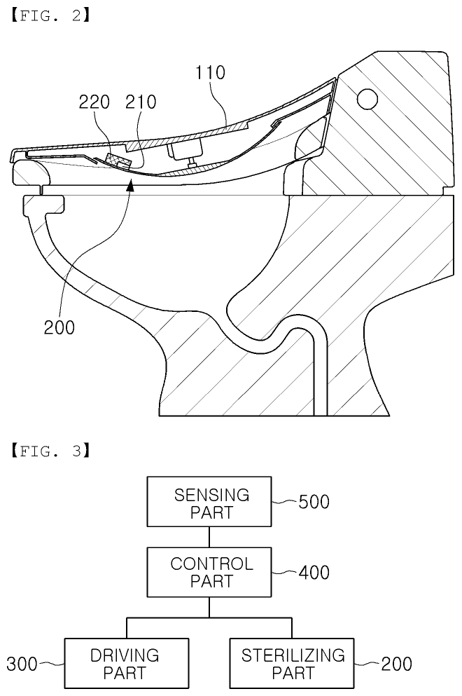

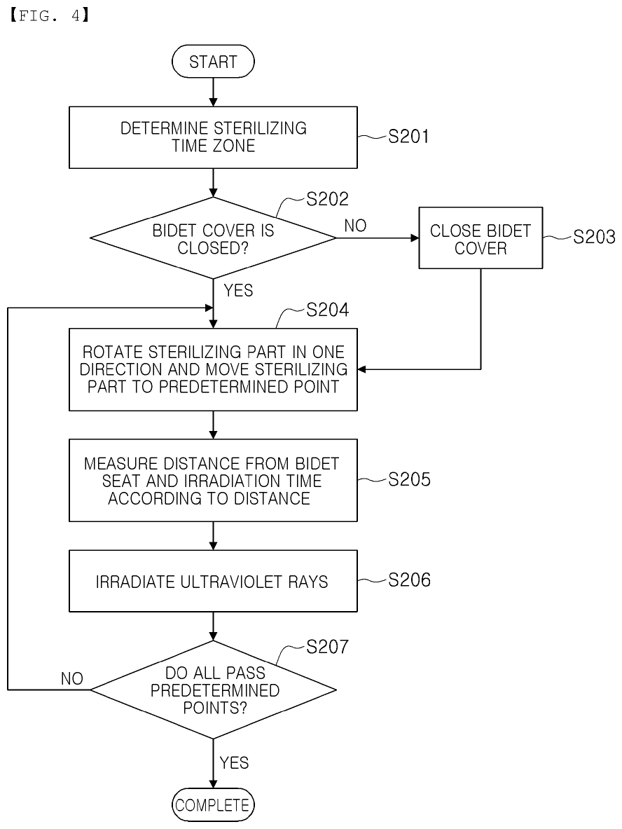

FIG. 1 is a front view of a bidet having a bidet seat sterilizing function according to an embodiment of the present disclosure, FIG. 2 is a side view of a bidet having a bidet seat sterilizing function according to an embodiment of the present disclosure, and FIG. 3 is a configuration diagram of a bidet having a bidet seat sterilizing function according to an embodiment of the present disclosure.

Referring to FIGS. 1 to 3, a bidet having a bidet seat sterilizing function according to an embodiment of the present disclosure may include a bidet seat 100, a bidet cover 110, a sterilizing part 200, a driving part 300, a control part 400, and a sensing part 500.

The bidet seat 100 may be disposed on an upper portion of a toilet seat, and a user may be seated thereon.

The bidet cover 110 may be rotatably connected to one side of the bidet seat 100 and may open and close the bidet seat 100.

The sterilizing part 200 may be installed on a bidet seat-facing surface of the bidet cover 110. When the bidet cover 110 covers the bidet seat 100, the bidet seat 100 may be sterilized.

The sterilizing part 200 may rotate in one direction.

According to an embodiment of the present disclosure, the sterilizing part 200 may include a distant sensor 210 for measuring a distance from the bidet seat 100 and a light irradiation part 220 for irradiating ultraviolet rays toward the bidet seat 100.

The distant sensor 210 and the light irradiation part 220 may be attached to each other and installed in the sterilizing part 200.

According to an embodiment of the present disclosure, the light irradiation part 220 may include an ultraviolet light emitting diode (UVLED) for irradiating UV-C in a range of 200 to 280 nm.

The driving part 300 may drive the bidet seat 100 and the sterilizing part 200.

The driving part 300 may include a driving motor for opening or closing the bidet seat 100 and a damper for rotating the sterilizing part 200, and may be installed on one side of the bidet seat 100.

The sensing part 500 may sense whether or not a user uses a bidet.

The sensing part 500 may be installed on a lower surface of the bidet seat 100, and may sense whether or not a user has used the bidet determining whether or not a certain amount of pressure is applied to the bidet seat 100. The sensing part 500 may include a switch, a pressure sensor and the like.

The control part 400 may control the sterilizing part 200 and the driving part 300.

Specifically, the control part 400 may be provided such that the bidet cover 110 covers the bidet seat 100, and may sterilize the bidet seat 100.

According to an embodiment of the present disclosure, the control part 400 may determine a sterilizing time zone by analyzing a usage pattern of a user for a predetermined period, based on whether or not the user uses the bidet sensed by the sensing part 500. When the determined sterilizing time zone is reached, the control part 400 may be provided such that the bidet cover 110 covers the bidet seat 100, and may operate the sterilizing part 200. Here, the predetermined period may be determined in units of days, weeks, months, and the like.

According to another embodiment of the present disclosure, the control part 400 may rotate the sterilizing part 200 to a predetermined point in one direction, may measure a distance from the bidet seat 100 and determine an irradiation time according to the measured distance, and may irradiate ultraviolet rays toward the bidet seat 100 for the determined irradiation time to sterilize the bidet seat 100.

The control part 400 may include at least one processing unit and a memory.

Here, the processing unit may include a central processing unit (CPU), a graphics processing unit (GPU), a microprocessor, an application specific integrated circuit (ASIC), a field programmable gate array (FPGA), and may have a plurality of cores. The memory may be a volatile memory, a non-volatile memory, or a combination thereof.

A control operation in the control part 400 will be described in more detail below with reference to FIG. 4.

FIG. 4 is a flow chart of the control part 400 of FIG. 3.

Referring to FIG. 4, first, the control part 400 may determine a sterilizing time zone (S201). Specifically, the sterilizing time zone may be determined to a time at which a user does not use the bidet by analyzing a usage pattern of the user.

Next, when the sterilizing time zone is reached, it is determined whether or not the bidet cover 110 is closed (S202). If the bidet cover 110 is opened, the bidet cover 110 may be closed by using a damper (S203).

The control part 400 may rotate the sterilizing part 200 in one direction and may move the distant sensor 210 and the light irradiating part 220 to a predetermined point (S204).

Specifically, an irradiation angle of the light irradiation part 220 may be determined to 55° to 120°, and each point may be determined to an interval of 30° to 60°.

The control part 400 may measure a distance from the bidet seat 100 using the distant sensor 210 and determine an irradiation time according to the distance (S205), and may irradiate ultraviolet rays for the determined irradiation time (S206).

Specifically, according to the following Table 1, the irradiation time may be determined according to a distance such that the sterilization is performed to be 99% or more.

TABLE 1

| | (Horizontal) distance from a light source [mm] | | |
|---|---|---|---|
| Irradiation time [min] | 0 | 55 | 110 |
| 0 | | | |
| 3 | 89.337% | | |
| 5 | | 80.8758% | |
| 10 | 99.994% | 98.8741% | |
| 15 | 100.000% | | |
| 20 | 100.000% | 99.9509% | |
| 30 | 100.000% | 99.9991% | |
| 45 | | 99.9998% | 98.0696% |
| 60 | | | 99.7151% |
| 75 | | | 99.9619% |

Next, the control part 400 repeats steps S204 to S206 until it passes all predetermined points, and may complete an sterilizing operation if it passes all the predetermined points (S207).

While exemplary embodiments have been shown and described above, it will be apparent to those skilled in the art that modifications and variations could be made without departing from the scope of the present disclosure as defined by the appended claims.

1. A bidet having a bidet seat sterilizing function, comprising:
a bidet seat, disposed on an upper portion of a toilet seat, allowing a user to be seated thereon;
a bidet cover for opening and closing the bidet seat;
a sterilizing part installed on the bidet seat-facing surface of the bidet cover and sterilizing the bidet seat when the bidet cover covers the bidet seat;
a driving part for driving the bidet seat and the sterilizing part; and
a control part for controlling the sterilizing part and the driving part.

2. The bidet having the bidet seat sterilizing function of claim 1, further comprising a sensing part for sensing whether or not a user uses the bidet.

3. The bidet having the bidet seat sterilizing function of claim 2, wherein the control part determines a sterilizing time zone by measuring a usage pattern of a user for a predetermined period, based on whether or not a user uses the bidet, sensed by the sensing part, is provided such that the bidet cover covers the bidet seat, and operates the sterilizing part.

4. The bidet having the bidet seat sterilizing function of claim 1, wherein the sterilizing part comprises a distant sensor for measuring a distance from the bidet seat; and
a light irradiating part for irradiating ultraviolet rays.

5. The bidet having the bidet seat sterilizing function of claim 4, wherein the control part is provided such that the bidet cover covers the bidet seat, and operates the sterilizing part.

6. The bidet having the bidet seat sterilizing function of claim 5, wherein the control part rotates the sterilizing part to a predetermined point in one direction, determines an irradiation time according to the measured distance by measuring the distance from the bidet seat, and irradiates ultraviolet rays toward the bidet seat for the determined irradiation time.

7. The bidet having the bidet seat sterilizing function of claim 4, wherein the light irradiating part comprises a ultraviolet light emitting diode (UVLED) for irradiating UV-C in a range of 200 to 280 nm.
